(19) United States
(12) Patent Application Publication
(10) Pub. No.: US 2004/0186104 A1
(43) Pub. Date: Sep. 23, 2004

(54) Novel pyridinone and related heterocyclic derivatives

(76) Inventors: Magnus Johansson, Molndal (SE); Asa Rosenquist, Linkoping (SE); Graeme Semple, Billdal (SE)

Correspondence Address: WHITE & CASE LLP, PATENT DEPARTMENT, 1155 AVENUE OF THE AMERICAS, NEW YORK, NY, 10036, US

(21) Appl. No.: 10/476,897

(22) PCT Filed: Apr. 29, 2002
(86) PCT No.: PCT/SE02/00839

(30) Foreign Application Priority Data
May 4, 2001 (SE) 0101579-1

(51) Int. Cl. C07D 217/22; A61K 31/53; A61K 31/497; A61K 31/513

(52) U.S. Cl. 514/242; 514/252.1; 514/269; 514/345; 544/182; 544/319; 546/141; 546/300; 514/309

(57) ABSTRACT

The present invention relates to a compound of formula (1), (2) or (3) having the following structures: Formula 1, 2, 3 wherein X, Y, and Z are independently C or N; A is a direct bond, CH2 or NH;B is a direct bond or NH; n=0-2; R1 is H, optionally substituted C₁₋₄191 alkyl, C₃₋₇191 cycloalkyl, halogen, cyano, nitro, aryl, or alkylaryl; R2 is H, C₁₋₄191 alkyl, or alkoxy C₁₋₄191 alkyl;or R1 and R2 are taken together to form an unsaturated 6-membered aromatic or heterocyclic ring containing one or two heteroatoms fused to the pyridone; R3 is a direct bond, H, C₁₋₄191 alkyl, substituted C₁₋₄191 alkyl, C₃₋₇191 cycloalkyl, aryl or alkylaryl; R4 is a direct bond or H; R5 is C₁₋₄191 alkyl or aryl; R6 and R7 are independently H or C₁₋₄191 alkyl; R8 and R9 are independently H, C₁₋₄191 alkyl, or tert-butoxycarbonyl or R8 and R9 are taken together with the nitrogen to which they are attached and form optionally, unsubstituted, substituted, fused or unsaturated 5-,6-,7-membered heterocycles containing one or two heteroatoms wherein said substituents are selected from the group consisting of hydroxyl, hydroxymethyl, carboxymethyl, carboxy, methoxy, and tert-butoxy;as (R)enantiomers, (S)-enantiomers or a racemate in the form of a free base or a pharmaceutically acceptable salt or solvate thereof.

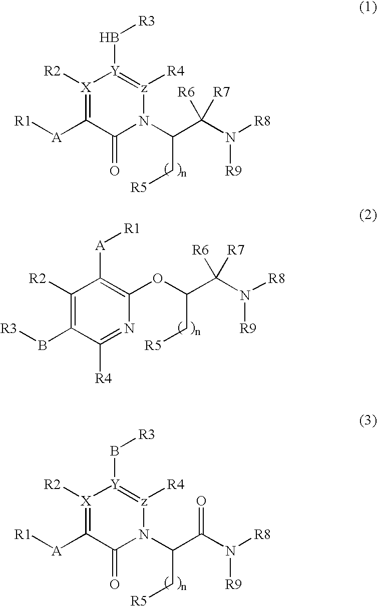

### FIELD OF THE INVENTION

This invention is concerned with novel pyridinone and related heterocyclic derivatives and to pharmaceutical compositions containing them, processes for their preparation, and their use in medicine. These compounds and pharmaceutical compositions exhibit significant agonist activity toward the κ-opioid receptor and are thus useful as analgesic, anti-inflammatory, diuretic, antitussive, anesthetic or neuroprotective agents, or as agents for treatment of stroke or functional bowel disorders in mammalian subjects, especially humans.

### BACKGROUND OF THE INVENTION

The kappa opioid receptor is a member of a family of related opioid receptors which include the mu (μ), delta (δ), kappa (κ), and sigma (σ) receptors as well as the more recently discovered ORL-1 or nociceptin receptor, which all appear to be present in the central and peripheral nervous system of many species, including man. The opioid receptors are all 7-transmembrane G-protein coupled receptors involved in pain transmission and nociception. The clinically used morphine-like analgesics, which act via non-specific μ-opioid receptor activation, have well-known side effects including respiratory depression, constipation and physical dependence liability. Compounds, which are κ-opioid receptor agonists, are known to act as analgesics with the advantage of being devoid of the serious side effects associated with the morphine-like analgesics. In addition to playing a role in antinociception, is has been showed that brain-penetrating κ-opioid receptor agonists may be useful for the treatment of stroke or as diuretic, antitussive or neuroprotective agents. As well as the well-characterized actions of opioids in the CNS, there is evidence for a role of opioid receptors in the periphery. It is now accepted that κ-opioid receptors are present in the peripheral terminals of primary afferent neurons and that the activation of these sites attenuates hyperalgesia, for example in rat models of arthritic pain. Therefore, a κ-opioid receptor agonist with a limited capability to penetrate the blood-brain barrier (BBB) may be effective in treating diseases where symptoms of pain and/or inflammation play an important role. Such a treatment would be devoid of CNS-related side effects associated with brain-penetrating κ-opioid receptor agonists.

### BRIEF DESCRIPTION OF THE INVENTION

The present invention provides compounds having κ-opioid receptor agonist activity. In its compound aspect, the present invention provides a compound of formula (1), (2) or (3) as (R)-enantiomers, (S)-enantiomers or a racemate in the form of a free base or a pharmaceutically acceptable salt or solvate thereof.

### DETAILED DESCRIPTION OF THE INVENTION

The compound of formula (1) has the following structure:

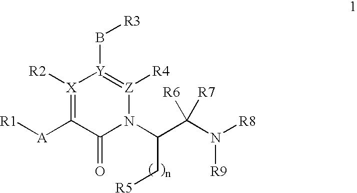

wherein

X, Y, and Z are independently C or N, where C is preferred

A is a direct bond, CH₂ or NH

B is a direct bond or NH

n=0-2, where n=0 is preferred

R1 is H, optionally substituted C₁₋₆ alkyl, C₃₋₇ cycloalkyl, halogen, cyano, nitro, aryl, or alkylaryl;

R2 is H, C₁₋₆ alkyl, or alkoxy C₁₋₆ alkyl or

R1 and R2 are taken together to form an unsaturated 6-membered aromatic or heterocyclic ring containing one or two heteroatoms fused to the pyridone;

R3 is H, C₁₋₆ alkyl, substituted C₁₋₆ alkyl, C₃₋₇ cycloalkyl, aryl or alkylaryl.

R4is H.

R5 is C₁₋₆ alkyl or aryl.

R6 and R7 are independently H or C₁₋₆ alkyl, where H is preferred.

R8 and R9 are independently H. C₁₋₆ alkyl, or tert-butoxycarbonyl or

R8 and R9 are taken together with the nitrogen to which they are attached and form optionally, unsubstituted, substituted, fused or unsaturated 5-,6-,7-membered heterocycles containing one or two heteroatoms wherein said substituents are selected from the group consisting of hydroxyl, hydroxymethyl, carboxymethyl, carboxy, methoxy, and tert-butoxy, as (R)-enantiomers, (S)-enantiomers or a racemate in the form of a free base or a pharmaceutically acceptable salt or solvate thereof.

The compound of formula (2) has the following structure:

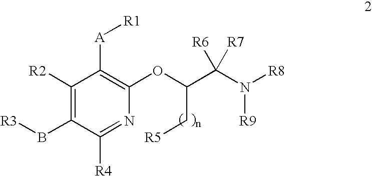

wherein

A, B, x, y, z, n, R1, R2, R3, R4, R5, R6, R7, R8 and R9 are as defined above, as (R)-enantiomers, (S)-enantiomers or a racemate in the form of a free base or a pharmaceutically acceptable salt or solvate thereof.

The compound of formula (3) has the following structure:

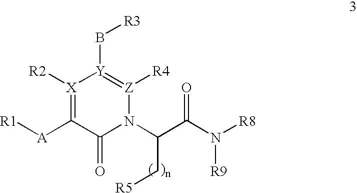

wherein

A, B, x, y, z, n, R1, R2, R3, R4, R5, R8, and R9 are as defined above; as (R)-enantiomers, (S)-enantiomers or a racemate in the form of a free base or a pharmaceutically acceptable salt or solvate thereof.

In the present context “halogen” may be fluoro, chloro, bromo or iodo.

In the present context “alkyl” may be a straight chain or branched, noncyclic or cyclic, unsaturated or saturated aliphatic hydrocarbon containing from 1 to 6 carbon atoms. Representative saturated straight chain alkyls include, methyl, ethyl, n-propyl, n-butyl, n-pentyl, n-hexyl and the like; while saturated branched alkyls include isopropyl, sec-butyl, isobutyl, tert-butyl, isopentyl and the like. Representative saturated cyclic alkyls include cyclopropyl, cyclobutyl, cyclopentyl, cyclohexyl and the like; while unsaturated cyclic alkyls include cyclopentenyl and cyclohexenyl and the like. Cycloalkyl may be a saturated or unsaturated (but not aromatic) carbocyclic ring containing from 3-7 carbon atoms, such as cyclopentane, cyclohexane, cycloheptane, cyclohexene, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

In the present context “alkoxy” may be an alkyl moiety attached through an oxygen bridge (ie. —O-alkyl) such as methoxy, ethoxy, and the like.

In the present context “alkylthio” may be an alkyl moiety attached through a sulfur bridge (i.e. —S-alkyl) such as methylthio, ethylthio, and the like.

In the present context “dialkylamino” may be an amino substituted with two alkyls.

The term “substituted” as used herein means any of the above groups wherein at least one hydrogen atom is replaced with a substituent. When substituted, “substituents” within the context of this invention include halogen, hydroxy, cyano, nitro, amino, alkylamino, dialkylamino, alkyl, alkoxy, alkylthio, haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐNR_{b}, —NRₐC(═O)OR_{b}, —NRₐSO₂R_{b}, —ORₐ, —C(═O)Rₐ, —C(═O)ORₐ, —C(═O)NRₐR_{b}, —OC(═O)NRₐR_{b}, SH, —SRₐ, —SORₐ, —S(═O)Rₐ, —OS(═O)Rₐ, —S(═O)ORₐ, wherin Rₐ and R_{b} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

In the present context “aryl” may be unsubstituted or mono-, di-, tri-, tetra-, or penta-substituted phenyl wherein said substituents are selected from the group consisting of halogen, optionally substituted C₁₋₄ alkyl,C₂₋₄ alkenyl, C₁₋₄ alkoxy-C₁₋₄ alkyl, substituted C₁₋₄ alkoxy-C₁₋₄ alkyl, C₁₋₄ alkylthio C₁₋₄ alkyl, hydroxy C₁₋₄ alkyl, C₃₋₇ cycloalkyl, alkyl or arylamide, carboxy-C₁₋₄alkyl, alkyl or arylsulfonamide, acetyl, formyl, carboxy, cyano, nitro, and amino; or optionally substituted phenyl, alkoxyphenyl, thiophenyl, furyl, naphtyl, 3,4-methylenedioxyphenyl, benzofuranyl, dibenzofuranyl, thianaphtyl, 1-thianthrenyl, 1-benzyl-2-benzimidazolyl, and ferrocenyl.

In the present context “alkylaryl” may be unsubstituted or mono-, di-, or tri-substituted C₁₋₄ alkyl phenyl wherein said substituents are selected from the group consisting of halogen, C₁₋₄ alkyl, substituted C₁₋₄ alkyl,C₂₋₄ alkenyl, C₁₋₄ alkoxy-C₁₋₄ alkyl, substituted C₁₋₄ alkoxy-C₁₋₄ alkyl, C₁₋₄ alkylthio C₁₋₄alkyl, hydroxy C₁₋₄alkyl, C₃₋₇ cycloalkyl, cyano, nitro, and amino; or optionally substituted 3,4-methylenedioxybenzyl.

Preferred individual compounds of the invention are:

1-[2-(4Morpholinyl)-1-phenylethyl]-3-(4-trifluoromethylphenyl)-2(1H)-pyridinone

1-[1-Phenyl-2-(1-pyrrolidinyl)-ethyl]-3-[4-(trifluoromethyl)phenyl]-2(1H)-pyridinone

4-Phenyl-2-[1-phenyl-2-(1-pyrrolidinyl)ethyl]-3(2H)-pyridazinone

1-[1-(2-Methoxyphenyl)-2-(1-pyrrolidinyl)-ethyl]-3-[4-(trifluoromethyl)phenyl)-2(1H)-pyridinone)

1-[1-Phenyl-2-(1-pyrrolidinyl)-ethyl]-3-bromo-2(1H)-pyridinone

1-[1-Phenyl-2-(1-pyrrolidinyl)-ethyl]-5-bromo-2(1H)-pyridinone

5-Bromo-[1-phenyl-2-(1-pyrrolidinyl)ethyl]-4(3H)-pyridinone

1-[1-Phenyl-2-(1-pyrrolidinyl)-ethyl]-3-(3,4-dichlorophenyl)-2(1H)-pyridinone

1-[1-Phenyl-2-(1-pyrrolidinyl)ethyl]-5-(3,4-dichlorophenyl)-2(1H)-pyridinone

5-(3,4-Dichlorophenyl)-[1-phenyl-2-(1-pyrrolidinyl)ethyl]-4(3H)-pyrimidinone

3-[(4-Methylphenyl)amino]-1-[1-phenyl-2-(1-pyrrolidinyl)ethyl]-2(1H)-pyridinone

Most preferred individual compounds of the invention are:

1-[1-Phenyl-2-(1-pyrrolidinyl)-ethyl]-3-(3,4-dichlorophenyl)-2(1H)-pyridinone

1-[1-Phenyl-2-(1-pyrrolidinyl)-ethyl]-3-[4-(trifluoromethyl)phenyl]-2(1H)-pyridinone

1-[1-(2-Methoxyphenyl)-2-(1-pyrrolidinyl)-ethyl]-3-[4-(trifluoromethyl)phenyl)-2(1H)-pyridinone).

Both organic and inorganic acids can be employed to form non-toxic pharmaceutically acceptable acid addition salts of the compounds of this invention. Illustrative acids are sulfuric, nitric, phosphoric, oxalic, hydrochloric, formic, hydrobromic, citric, acetic, lactic, tartaric, dibenzoyltartaric, diacetyltartaric, palmoic, ethanedisulfonic, sulfariic, succinic, propionic, glycolic, malic, gluconic, pyruvic, phenylacetic, 4-aminobenzoic, anthranilic, salicylic, 4-aminosalicylic, 4-hydroxybenzoic, 3,4-dihydroxybenzoic, 3,5-dihydroxybenzoic, 3-hydroxy-2-naphthoic, nicotinic, methanesulfonic, ethanesulfonic, hydroxyethanesulfonic, benzenesulfonic, prtoluenesulfonic, sulfanilic, naphthalenesulfonic, ascorbic, cyclohexylsulfamic, fumaric, maleic and benzoic acids. These salts are readily prepared by methods known in the art.

The preferred solvates of the compounds of this invention are the hydrates.

Pharmaceutical for Tions

In a second aspect the present invention provides a pharmaceutical formulation comprising as active ingredient a therapeutically effective amount of the compound of formula 1 as an enantiomer or a racemate in the form of a free base or a pharmaceutically acceptable salt or solvate thereof, optionally in association with diluents, excipients or inert carriers.

According to the present invention the compound of the invention will normally be administered orally, rectally or by injection, in the form of pharmaceutical formulations comprising the active ingredient either as a free base or a pharmaceutically acceptable non-toxic acid addition salt, e.g. the hydrochloride, hydrobromide, lactate, acetate, phosphate, sulfate, sulfamate, citrate, tartrate, oxalate and the like in a pharmaceutically acceptable dosage form. The dosage form may be a solid, semisolid or liquid preparation. Usually the active substance will constitute between 0.1 and 99% by weight of the preparation, more specifically between 0.5 and 20% by weight for preparations intended for injection and between 0.2 and 50% by weight for preparations suitable for oral administration.

To produce pharmaceutical formulations containing the compound of the invention in the form of dosage units for oral application, the selected compound may be mixed with a solid excipient, e.g. lactose, saccharose, sorbitol, mannitol, starches such as potato starch, corn starch or amylopectin, cellulose derivatives, a binder such as gelatine or poly-vinylpyrrolidone, and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet can be coated with a polymer known to the person skilled in the art, dissolved in a readily volatile organic solvent or mixture of organic solvents. Dyestuffs may be added to these coatings in order to readily distinguish between tablets containing different active substances or different amounts of the active compound.

For the preparation of soft gelatine capsules, the active substance may be admixed with e.g. a vegetable oil or polyethylene glycol. Hard gelatine capsules may contain granules of the active substance using either the above mentioned excipients for tablets e.g. lactose, saccharose, sorbitol, mannitol, starches (e.g. potato starch, corn starch or amylopectin), cellulose derivatives or gelatine. Also liquids or semisolids of the drug-can be filled into hard gelatine capsules.

Dosage units for rectal application can be solutions or suspensions or can be prepared in the form of suppositories comprising the active substance in a mixture with a neutral fatty base, or gelatine rectal capsules comprising the active substance in admixture with vegetable oil or paraffin oil. Liquid preparations for oral application may be in the form of syrups or suspensions, for example solutions containing from about 0.1% to about 20% by weight of the active substance herein described, the balance being sugar and mixture of ethanol, water, glycerol and propylene glycol. Optionally such liquid preparations may contain colouring agents, flavouring agents, saccharine and carboxymethyl-cellulose as a thickening agent or other excipients known to the person skilled in the art.

Solutions for parenteral applications by injection can be prepared in an aqueous solution of a water-soluble pharmaceutically acceptable salt of the active substance, preferably in a concentration of from about 0.1% to about 10% by weight. These solutions may also contain stabilising agents and/or buffering agents and may conveniently be provided in various dosage unit ampoules.

Suitable daily doses of the compound of the invention in therapeutical treatment of humans are about 0.01-100 mg/kg bodyweight at peroral administration and 0.001-100 mg/kg bodyweight at parenteral administration.

Medical and Pharmaceutical Use

The pyridinone related heterocyclic derivatives of the present invention of formulae (1), (2), and (3) exhibit significant agonist activity toward the κ-opioid receptor and are thus useful as analgesic, anti-inflammatory, diuretic, antitussive, anesthetic or neuroprotective agents, or as agents for treatment of stroke or functional bowel disorders, for the treatment of mammalian subjects especially humans.

General Synthesis

The κ-opioid receptor agonists of formula (1) of the present invention may be prepared by a variety of synthetic methods.

The compounds of formula (1) may be prepared by reduction of the heterocyclic amide compounds (2) followed by deprotection of any optionally used protecting groups in R5, R8, and R9 as described in Preparation Method A-I.

Preparation Method A-I:

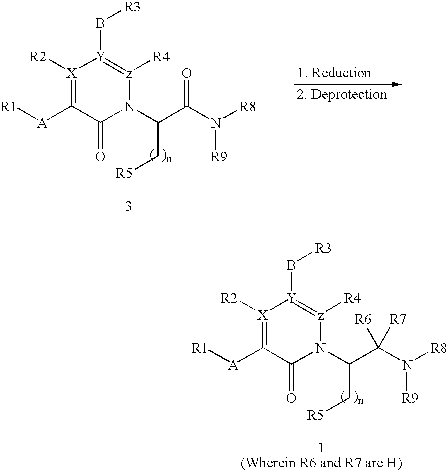

In Preparation Method A-I reduction methods known to those skilled in the art may be used. For example, the heterocyclic amide compound (3) may be reduced with borane dimethylsulfide. The reaction may be carried out in a suitable reaction-inert solvent, such as dry tetrahydrofuran, at a temperature from 0° C. to 25° C., preferably at 25° C., for 1 hour to 24 hours. Alternatively, the reduction of the heterocyclic amide compound (3) may be lo carried out by conversion of the amide compound (3) to the corresponding thioamide using the Lawesson's reagent followed by reduction with Raney nickel. The conditions for this method may be appropriately chosen by those skilled in the art.

In the case where protecting groups are used in R5, R8 or R9, the protecting groups are removed by the appropriate method for the particular protecting group chosen. Thus, a typical protecting group tert-butyldimethylsilyl. This may be removed by treatment with fluoride ion from, for example, tetra-n-butylammonium fluoride, aqueous hydrogen fluoride, or borontrifluoride, preferably from tetra-n-butylammonium fluoride. Further used protecting groups are the methyl- and tert-butyl-esters. The methylester may be removed by base-catalyzed hydrolysis. Appropriate basic catalysts are sodium hydroxide, lithium hydroxide, barium hydroxide, or potassium hexamethylsilanoate, preferably, lithium hydroxide. The tert-butylester may be removed by acid-catalyzed hydrolysis.

Appropriate acid catalysts are formic acid, trifluoroactetic acid, acetic acid, hydrogen bromide, or p-toluenesulfonic acid, preferably trifluoroacteic acid.

In an alternative method, the compounds of formula (1) may be obtained by alkylation of the heterocyclic amide compounds (4) under Mitsonobu- or under basic- conditions followed by removal of any optionally used protecting groups in R5, R8, or R9 according to Preparation Method A-II.

The compounds of formula (2) may also be obtained by alkylation of the heterocyclic amide compounds (4) under the Mitsonobu conditions described in Preparation Method A-II.

Preparation Method A-II:

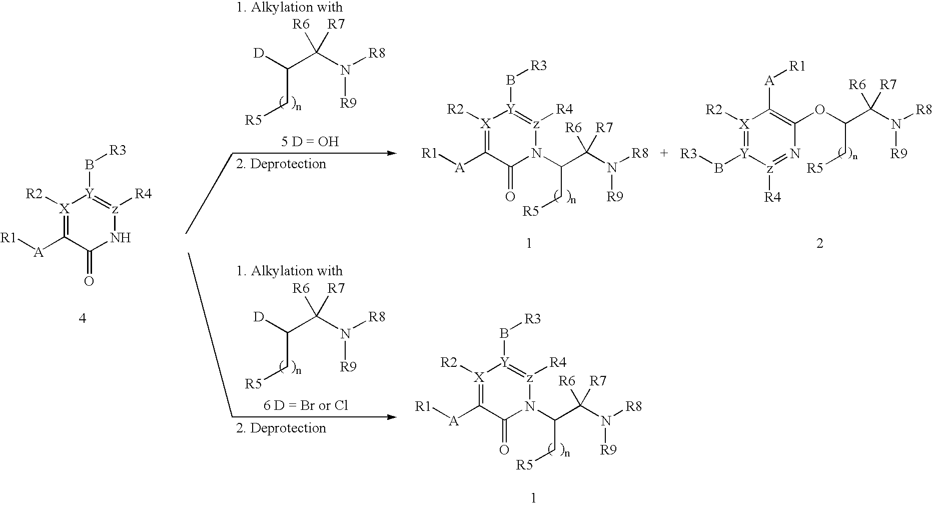

The heterocyclic amide compounds (4) may be alkylated under Mitsonobu conditions, which comprises the reaction of the amide compound (4) with an alkylalcohol (5) (wherein R5, R6, R7, R8, and R9 are as already defined), triphenylphosphine or tributylphosphine, and either diethylazodicarboxylat, diisopropylazodicarboxylat or 1,1 ′-azobis(N,N-dimethylformamide), preferably tributylphosphine and 1,1′-azobis(N,N-dimethylformamide), in a suitable reaction-inert solvent. Suitable reaction-inert solvents include, for example, tetrahydrofuran, dimethoxyethane, diethylether, or hexane. The reaction may be carried out at a temperature from 0° C. to 25° C., preferably at 25° C., for 30 minutes to 24 hours. Finally, in the case where a protecting groups is used in R5, R8 or R9, the protecting groups are removed by the appropriate method for the particular protecting group chosen using the same reaction conditions as described in Preparation Method A-I.

The compounds of formula (2) may be obtained by alkylation of the heterocyclic amide compounds (4) under the Mitsonobu conditions described in Preparation Method A-II. Depending on the structure of the amide compounds (4) and the alkylalcohol (5) the N-alkylated products of formula (1) and the O-alkylated products of formula (2) are obtained in ratios from 100:1 to 10:1. The formation of the O-alkylated products of formula (2) is favored by the use of triphenylphosphine and diethylazodicarboxylat.

In addition, the heterocyclic amide compounds (4) may be obtain ed by alkylation under basic conditions. For example, the compounds of formula (1) may be obtained by alkylation of the amide compounds (4) with an alkylhalide (6) (wherein R5, R6, R7, R8, and R9 are as already defined). In a typical procedure the amide compound (4) is reacted with a base such as sodium, sodiumhydride, sodiumhydroxide, potassiumhydroxide, potassiumcarbon ate, or potassiumhexamethyldisilazane, preferable sodiumhydride, followed by an alkylbalide (4) in a reaction-inert solvent. Suitable inert-reaction solvents include, for example, dimethylformamide, dimethylsulfoxide, acetonitrile, or tetrahydrofuran. The reaction may be carried out at a temperature from 0° C. to 150° C., preferably from 0° C. to 80° C., for 30 minutes to 48 hours.

The amide compounds (4) are either commercially available, known, or prepared according to known procedures as described in the literature, for example, J. Chem. Soc. Perkin Trans. 1985, 1, 1627; J. Med Chem. 1981, 24, 1475; J. Heterocyclic Chem. 1986, 23, 1071.

Further, the compounds of formula (1) may be obtained by palladium-catalyzed coupling of the heterocyclic bromide compounds (7) or (8) with aryl boronic acids, respectively, according to Preparation Method A-III.

Preparation Method A-III:

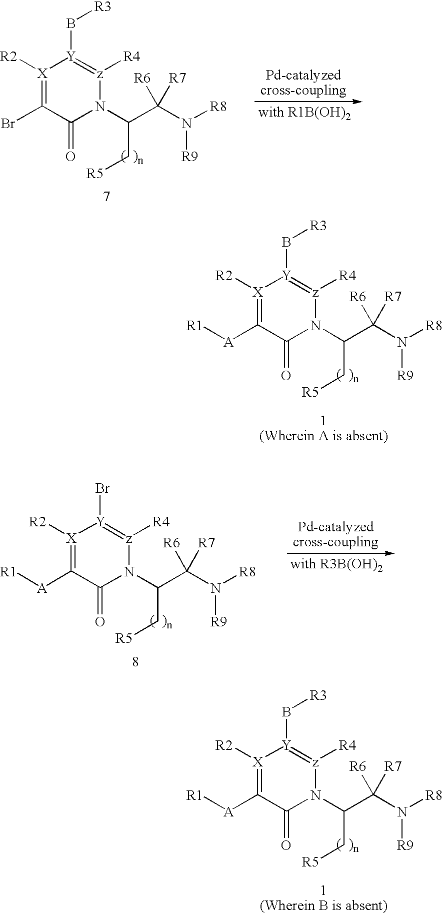

In a typical procedure the bromide compounds (7) or (8) may be reacted with aryl boronic acids R1B(OH)₂ or R3B(OH)₂, respectively, (wherein R1 and R3 are as already defined) in the presence of a suitable palladium-source and base. Suitable palladium-sources are Pd(OAc)₂, Pd(PPh₃)₄, PdCl₂(PPh₃)₂, Pd₂(dba)₃, or PdCl₂(dppf), preferable Pd(OAc)₂ or Pd(PPh₃)₄. A variety of bases may be used, for example, aqueous-sodiumcarbonate, -potassiumcarbonate, -sodiumhydroxide, -bariumhydroxide, -cesiumfluoride, or -potassiumphosphate. Many bases may also be used under non-aqueous conditions, for example, potassiumphosphate, ceasuimcarbonate, cesiumfluoride, potassiumfluoride, triethylarnine, and diisopropylethylamine. Preferably aqueous potassiumcarbonate is used. The reaction is carried out in a reaction-inert solvent, for example, dioxane, acetone, acetonitril, dimethoxyethane, toluene, or dimethylfomamide, preferably acetone, under inert atmosphere at a temperature from 25° C. to 120° C., preferably from 50° C. to 110° C., for 1 hour to 48 hours.

The bromide compounds (7) and (8) are synthesised from commercially available 2-hydroxy pyridine or 3H-pyrimidin-4-one according to methods described in the literature, for example, Organic Synthesis, Wiley: New York, 1973, Collect. Vol. V, 346; J. Am. Chem. Soc. 1982, 104, 4141; J. Org. Chem. 1985, 50, 3073; and J. Chem. Soc. 1955, 3478. This is followed by alkylation with an alkylalcohol compound (5) or alkylhalide compound (6) or using the same reaction conditions as described in Preparation Method A-II.

The compounds of formula (1), wherein A is NH may be obtained by palladium-catalyzed amination of the heterocyclic bromide compounds (7), according to Preparation Method A-IV. This preparation method may also be used to obtain the compounds of formula (1) wherein B is NH starting from the heterocyclic bromide compounds (8).

Preparation Method A-IV:

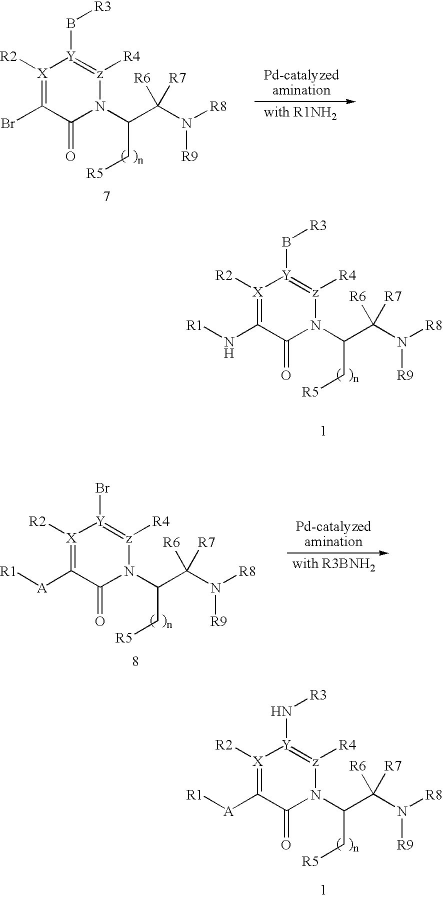

In a typical procedure the heterocyclic bromide compounds (7) or (8) may be reacted with amines R1NH₂ or R3NH₂, respectively, (wherein R1 and R3 are as already defined) in the presence of a suitable palladium-source, chelating ligand, and base. The skilled persons may properly choose the conditions employed for this method. Preferably, Pd(OAc)₂ or Pd₂(dba)₃ are used as palladium-source, BINAP is used as chelating agent, and potassium-tert-butoxide or cesiumcarbonate is used as base. The reaction is carried out in a reaction-inert solvent, for example, dioxane, acetone, acetonitril, dimethoxyethane, toluene, or dimethylfomamide, preferably dioxane, under inert atmosphere at a temperature from 25° C. to 120° C., preferable from 50° C. to 85° C., for 1 hour to 48 hours. The bromide compounds (7) and (8) are from the same source as described above in Preparation Method A-III.

The compounds of formula (1), wherein R6 and R7 are H and R8 and R9 are taken together with the nitrogen to form a 5, 6, or 7-membered heterocyclic ring optionally substituted with a hydroxy group, may be obtained by a solid-phase reaction procedure as described in Preparation Method A-V, Part I-IV.

Preparation Method A-V (Part I)

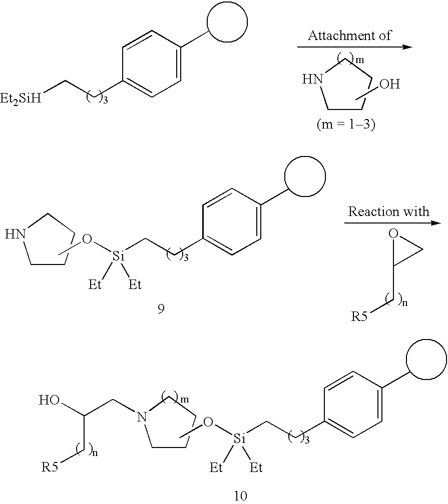

In Preparation Method A-V general methodologies for solid-phase chemistry known to those skilled in the art should be used. In a typical procedure the alcohol compounds may be attached to the solid-support through a butyl diethylsilane linker (Part I). Preferably, the solid-support is treated with the alcohol compound in a suitable reaction-inert solvent such as NMP using RhCl (PPh₃)₃. The reaction may be carried out at a temperature from 25° C. to 60° C., preferably 60° C., for 30 minutes to 12 hours. The support-bound amine (9) is then reacted with epoxide compounds to give the support-bound alcohol (10). The reaction may be carried out in only the epoxide compounds or in a suitable reaction-inert solvent, for example dimethylformamide or NMP, at a temperature from 25° C. to 100° C., preferably from 60° C. to 80° C., for 1 hour to 12 hours.

Preparation Method A-V (Part II):

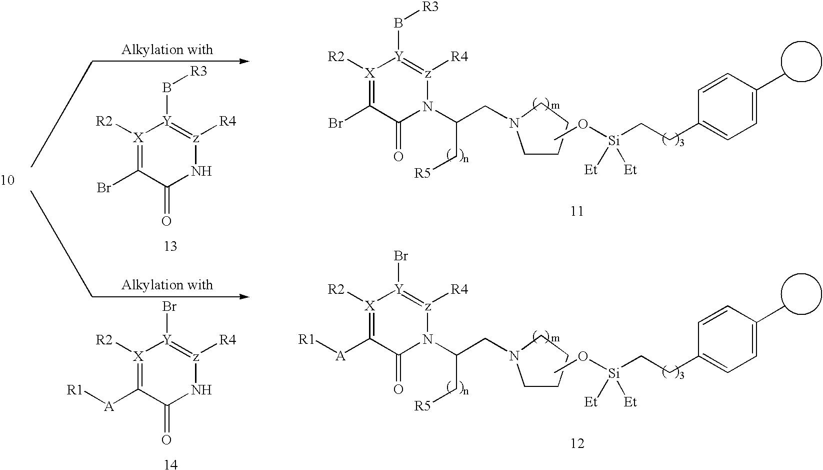

The support-bound alcohol (10) is then alkylated with the heterocyclic bromide compounds (13) or (14), respectively, to give the corresponding support-bound heterocyclic bromides (11) or (12), (Part II) under Mitsonobu conditions as described in Preparation Method A-III.

Preparation Method A-V (Part III):

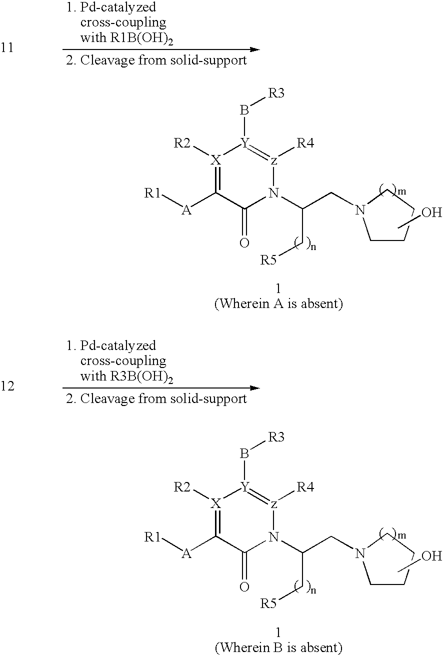

The palladium-catalyzed coupling of the support-bound heterocyclic bromide compounds (11) and (12) with aryl boronic acids R1B(OH)₂ or R3B(OH)₂, respectively, (wherein R1 and R3 are as already defined) may be performed under the same conditions described in Preparation Method A-II. Preferably, Pd(PPh₃)₄ is used as palladium-source and potassium carbonate as base. The compounds of formula (1) may finally be cleaved from the solid-support by fluoride ions from, for example, tetra-n-butylammoniumfluoride in pyridine and MeOSiMe₃, or preferably by-acid catalyzed hydrolysis, using acetic acid in tetrahydrofuran and water.

Preparation Method A-V (Part IV):

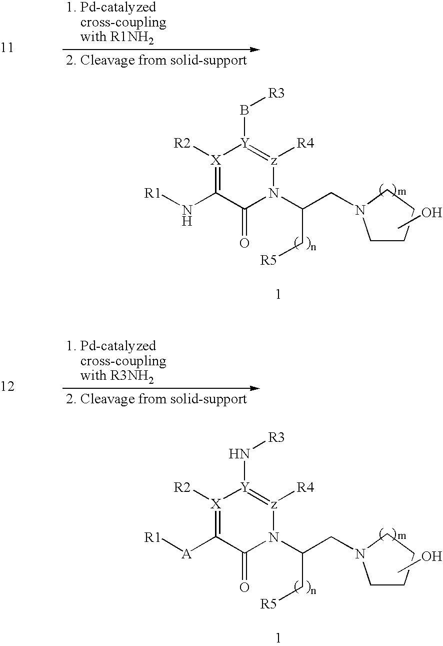

Further, the palladium-catalyzed amination of the support-bound bromide compounds (11) and (12) with amine compounds R1NH₂ or R3NH₂, respectively, (wherein R1 and R3 are as already defined) (Part IV) may be performed under the same conditions described in Preparation Method A-IV. The compounds of formula (1) are finally cleaved from the solid-support as described above in Preparation method A-V, Part III.

In the present invention the compounds of formula (3), used in Preparation Method A-I, may be obtained by alkylation of the heterocyclic amide compounds (4) followed by removal of the protecting group P and coupling of the corresponding carboxylic acid compound (15) with amines as described in Preparation Method B-I.

Preparation Method B-I:

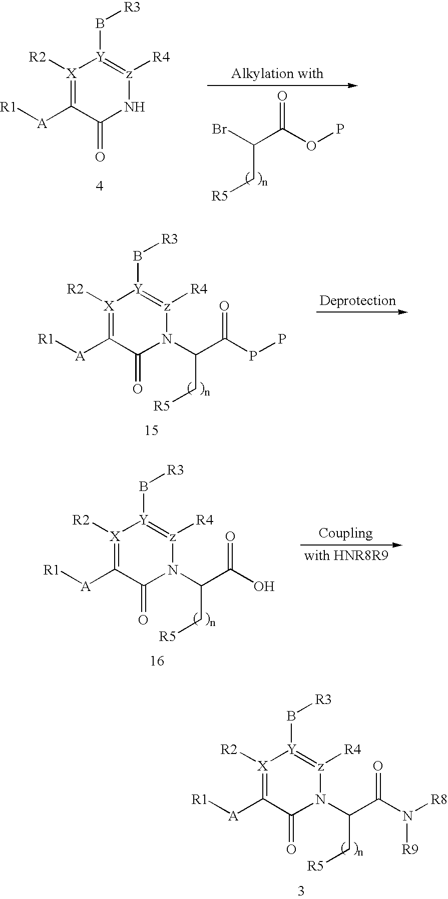

In this method the heterocyclic amide compounds of formula (4) is alkylated with an alkylating agent using standard alkylating techniques known to those skilled in the art. Preferable, the alkylation conditions described above in Preparation Method A-II are used. The protecting group P is removed by an appropriate method for the particular protecting group chosen. Thus, typical protecting groups methyl or ethyl. These may be removed by base-catalyzed hydrolysis. Appropriate basic catalysts are sodium hydroxide, lithium hydroxide, barium hydroxide, or potassium hexamethylsilanoate, preferable, lithium hydroxide. The carboxylic acid compound (16) is then reacted with an amine using standard coupling techniques known to those skilled in the art. In this procedure the carboxylic acid compound (16) may be reacted with a coupling reagent followed by the amine optionally in the presence of base. Suitable coupling reagents are TBTU, PyBOP, DCC and HOAt, or HATU. Suitable inert-reaction solvents include, for example dimethylformamid or dichloromethane. The reaction may be carried out at temperature from −10° C. to 100° C., preferably at 0° C. to 25° C., for 2 h to 45 hours. The compounds of formula (3) may also be obtained from the carboxylic acid compounds (16) by other methods, for example, by conversion to the corresponding acid chloride and further reaction with an amine. The conditions for this method may be appropriately chosen by those skilled in the art. The amide compounds (4) are from the same source as described above in Preparation method A-II.

In the present invention, the alkylalcohol compounds (5) and alkylhalide compounds (6) and, used in preparation method A-2, may be obtained by the following Preparation Method C-I.

Preparation Method C-I.

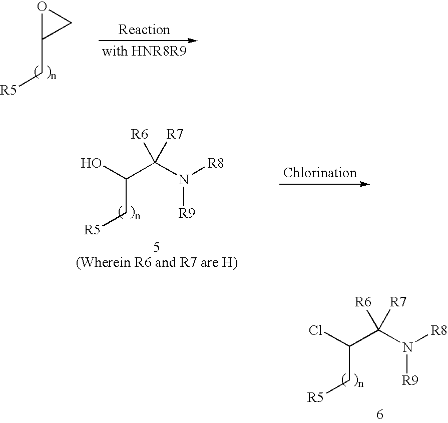

In this method the alkylalcohol compounds (5) may be obtained from the corresponding epoxide that is reacted with the amine compounds HNR8R9 (wherein R8 and R9 are as already defined). The reaction may be carried out in only the amine compound or in a suitable reaction-inert solvent at a temperature from 25° C. to 100° C., preferably from 60° C. to 80° C., for 1 hour to 12 hours. Suitable reaction-inert solvents include, for example, methanol, ethanol, propanol or acetonitrile. The alkylhalide compounds (6) is then obtained by chlorination under standard conditions known to those skilled in the art. The epoxide compounds are either commercially available, known, or prepared according to known procedures as described in the literature.

Alternatively, alkylalcohol compounds (5) may be obtained by substitution of the α-halo ketone compounds, followed by reduction, according to Preparation Method C-II.

Preparation Method C-II.

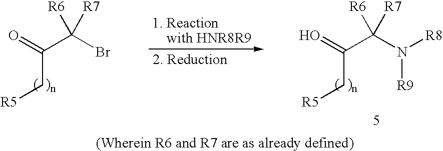

In a typical procedure, the α-halo ketone is reacted with amines HNR8R9 (wherein R8 and R9 are as already defined) in a suitable reaction-inert solvent, for example tetrahydrofuran, followed by reduction using NaBH₄ as reducing agent in a suitable solvent such as methanol or ethanol. This reaction may be carried out at a temperature from 0° C. to 25° C., for 30 minutes to 24 hours.

The α-halo ketone compounds are commercially available, known, or prepared according to known procedures as described in the literature.

The compounds of formula (1), (2), and (3) and the intermediates shown in the above Preparation Methods may be isolated and purified by conventional procedures such as recrystallization or chromatographic purification.

As compounds of this invention possess at least two asymmetric centres, They are capable of occurring in various stereoisomeric forms or configurations. Hence, the compounds may exist in separated (+)- and (−)-optically active forms as well as mixtures thereof. The present invention includes all such forms within the scope. Individual isomers may be obtained by known methods, such as stereoselective reactions or chromatographic separation methods in the preparation of the final products or its intermediates.

### EXAMPLES

The present invention is illustrated by the following examples. However, it should be understood that the invention is not limited to the specific details of these examples. ¹H and ¹³C nuclear magnetic resonance spectra (NMR) were measured in CDCl₃, DMSO-d₆ or MEOH-d₄ by Bruker NMR spectrometers, unless otherwise indicated the peak positions are expressed in parts per million (PPM) downfield from tetramethylsilane. The peaks are denoted as follows: s, singlet; d, doublet; t, triplet; m, multiplet; Br, broad. Column chromatography was performed using silica gel 60 (0.040-0.063 mm, Merk).

### PREPARATIONS

### Preparation 1

### 3-(4-Trifluoromethylphenyl)pyridine

To solution of 4-bromo-(trifluoromethyl)benzene (12 g, 54 mmol) in dry THF (40 mL) was magnesium turnings (1.19 g, 49 mmol) added and the mixture was heated under reflux under a nitrogen atmosphere until no magnesium remained. After cooling to room temperature the reaction mixture was added to a solution of 3-bromopyridine (7.11 g, 45 mmol) and bis(triphenylphosphine)nickel (II) chloride (0.52 g, 1 mmol) in dry THF (60 mL) cooled in an ice-water bath. The temperature was kept below 10° C. during the addition. When the addition was complete, the reaction mixture was allowed to reach room temperature and then stirred for 36 h. The resulting mixture was poured into aqueous HCl (1 M) and washed with ether. The aqueous portion was neutralized with aqueous NaOH and extracted twice with EtOAc. The combined organic extracts were washed with brine, dried (MgSO₄) and concentrated. The product was purified by column chromatography (EtOAc:Heptane; 1:3) to provide 3-(4-trifluoromethylphenyl)pyridine (3.1 g, 30%) as a fluffy white solid.

¹H NMR (300 MHz, CDCl₃) δ 7.13 (ddd, J=7.7, 4.7, 0.7 Hz, 1H), 7.73 (dd, J=16.8, 8.7, 4H), 7.91 (m, 1H), 8.67 (dd, J=5.0, 1.7 Hz, 1 H), 8.88 (d, J=1.7 Hz, 1H).

### Preparation 2

### 3-(4-Trifluoromethylphenyl)pyridine 1-oxide

A mixture of 3-(4-trifluoromethylphenyl)pyridine (3.0 g, 13 mmol), acetic acid (30 mL) and 35% hydrogen peroxide (3 mL) was stirred at 70-80° C. for 18 h. The mixture was cooled and concentrated. The residue was azeotroped twice with toluene and purified by crystallization from Et₂O and heptane to give 3-(4-trifluoromethylphenyl)pyridine 1-oxide (2.2 g, 68%) as a white solid.

¹H NMR (400 MHz, CDCl₃) δ 7.44 (dd, J=7.8, 6.3 Hz, 1H), 7.56 (d, J=8.3 Hz, 1H), 7.69 (d, J=8.8 Hz, 2H), 7.79 (d, J=8.3 Hz, 2 H), 8.33 (d, J=6.3 Hz, 1H), 8.60 (t, J=1.5 Hz, 1 H).

### Preparation 3

### 3-(4-Trifluoromethylphenyl)-2(1H)-pyridinone

The 3-(4-trifluoromethylphenyl)pyridine 1-oxide (2.0 g, 8.3 mmol) was taken up in acetic anhydride (100 mL) and heated under reflux temperature for 4 h. The mixture was cooled and concentrated and the residual oil was taken up in ethanol (50 mL), treated with decolorizing charcoal, and filtered through celite. The mixture was partially evaporated to induce crystallization. The resultant white crystals of 3-(4-trifluoromethyl]phenyl)-2(1H)-pyridinone (0.97 g, 49%) were collected by filtration. A second crop of the product (0.16 g, 8%) was obtained by crystallization from EtOH and Et₂O.

¹H NMR. (300 MHz, DMSO-d₆) δ 6.32 (t, J=6.7 Hz, 1H), 7.46 (dd, J=6.4, 2.0 Hz, 1 H), 7.74 (m, 3 H), 7.95 (d, J=8.1 Hz, 2 H), 11.90 (b, 1 H).

### Preparation 4

### 5-Ethoxydihydro-3-phenyl-2(3H)-furanone

To a mixture of diisopropylamine (6.7 mL, 49.0 mmol) in THF (50 mL) was a solution of n-butyl lithium in hexanes (100 mL, 1.6 M) added at −78° C. After stirring at −78° C. for 10 min a solution of phenyl acetic acid (5.95 g, 44.0 mmol) in THF (20 mL) was added. The cold bath was removed and the reaction mixture allowed to reach room temperature over 40 min before bromoacetaldehyde diethylacetal (13.4 mL, 89.0 mmol) was added and the reaction mixture was heated under reflux overnight. The reaction mixture was cooled, poured into water (400 mL) and extracted with Et₂O (50 mL). The aqueous portion was acidified with concentrated HCl and then extracted twice with EtOAc (100 mL). The combined EtOAc extracts were dried (Na₂SO₄) and concentrated. The resultant brown oil was distilled under reduced pressure (high vac pump, b.p. 10-115 C). Cyclisation occurs under these conditions to give the desired product. The mixture was taken up in EtOAc, washed with sat NaHCO₃ and brine, dried (Na₂SO₄), and concentrated to give pure 5-ethoxydihydro-3-phenyl-2(3H)-furanone (3.16 g, 35%) as a pale yellow oil.

### Preparation 5

### 4-5-Dihydro-4-phenyl-3(2H)-pyridazinone

To a mixture of 5-ethoxydihydro-3-phenyl-2(3H)-furanone (3.1 g, 15.0 mmol) in acetic acid (10 mL) and water (7 mL) was hydrazine hydrate (2.1 mL, 43.0 mmol) added and the mixture was heated under reflux for 2 h. The mixture was cooled to room temperature, partially concentrated to remove AcOH, and then poured into saturated NaHCO3 and extracted twice with EtOAc. The combined organic extracts were washed with brine, dried (Na₂SO₄), and concentrated to provide a yellow solid. Crystallization from EtOAc and heptane gave 4,5-dihydro-4-phenyl-3(2B)-pyridazinone (1.43 g, 55%) as a pale yellow solid.

¹H NMR (400 MHz, CDCl₃) δ 2.74-2.95 (m, 4 H), 3.74 (t, J=8.8 Hz, 1 H), 7.34 (m, 5 H), 8.64 (b, 1 H).

### Preparation 6

4-Phenyl-3(2H)-pyridazinone To a mixture of 4,5-dihydro-4-phenyl-3(2H)-pyridazinone (0.55 g, 3.0 mmol) in DMSO (12.5 mL) and water (0.5 mL) was NBS (1.50 g; 8.0 mmol) added at 0° C. The reaction mixture was allowed to attain room temperature and the resultant orange mixture was then stirred at room temperature for additional for 2 h before it was poured into saturated NaHCO₃. The product was extracted twice with EtOAc and the combined extracts washed with brine, dried (MgSO₄), and concentrated. Purification by column chromatography gave 4-phenyl-3(2H)-pyridazinone (0.065 g, 12%) as a white solid.

¹H NMR (400 MHz, MeOH-d4) δ 7.45 (m, 3 H), 7.54 (d, J=4.4 Hz, 1 H), 7.80,(m, 2 H), 7.96 (d, J=3.9 Hz, 1 H).

### Preparation 7

### 3-Bromo-2(1H)-pyridinone

To a mixture of 2-hydroxy pyridine (12.8 g, 135.0 mmol) in aqueous KBr (1 M, 200 mL) a solution of bromine (21.5 g, 135.0 mmol) in aqueous KBr (1 M, 300 mL) was added over a 10-min period. After stirring at room temperature for 24 h this mixture deposited a green precipitate of 3,5-dibromo-2(1H)-pyridinone that was removed by filtration. The remaining solution was concentrated to half volume to induce precipitation of the desired product. The greyish precipitate was collected by filtration and recrystallized from acetonitril to give 3-bromo-2(1H)-pyridinone (5.1 g, 22%) as a slightly yellow solid.

¹H NMR (400 MHz, DMSO-d₆) δ 6.13 (t, J=6:0 Hz, 1 H), 7.44 (dd, J=6.0,1.2 Hz, 1 H), 7.88 (dd, J=6.0, 1.2 Hz, 1 H), 12.5 (b, 1 H).

### Preparation 8

### 5-Bromo-2(1H)-pyridinone

A mixture of 2-hydroxy pyridine (4.8 g, 50.0 mmol) in acetic acid (50 mL) was treated with NBS (9.4 g, 53.0 mmol) at room temperature for 4 h. The mixture was concentrated, azeotroped twice with ethanol before the remaining solid was taken up in hot ethanol (100 mL). After cooling to room temperature the precipitate was removed by filtration and recrystallised from ethanol to provide 5-bromo-2(1H)-pyridinone (5.1 g, 59%) as a pale brown crystalline solid.

¹H NMR (400 MHz, DMSO-d₆) δ 6.34 (d, J=9.0 Hz, 1 H), 7.51 (dd, J=9.0, 2.4 Hz, 1 H), 7.54 (d, J=2.4 Hz, 1 H).

### Preparation 9

### 5-Bromo-4(3H)-pyrimidinone

5-Bromo-pyrimidine (7.9 g, 50.0 mmol) was taken up in acetone (50 mL) in a flask fitted with a reflux condenser at room temperature. A mixture of peracetic acid (20.8 mL of 32% solution in acetic acid) and concentrated sulfuric acid (2.8 mL) was added and the mixture stirred at room temperature. The reaction mixture became hot and began to reflux after 2 to 3 minutes. No heat was applied and the reaction was allowed to stir at ambient temperature for 2 h. During this time the mixture cooled down and a white precipitate was formed. The mixture was cooled on an ice-water bath and the product (7.7 g, 70% as hemisulfate salt) was collected by filtration. The hemisulfate salt was taken up in water and neutralized to pH 7 with 5M NaOH. The mixture was cooled to 4° C. for 1 h and the resultant precipitate was collected by filtration and washed with cold water-to provide 5-bromo-4(3H)-pyrimidinone (4.5 g, 52%) as a white solid.

¹H NMR (400 MHz, DMSO-d₆) δ 8.20 (s, 1 H), 8.30 (s, 1 H), 13.10 (b, 1 H).

### EXAMPLES

### Example 1

### 2-Oxo-α-phenyl-3-(4-trifluoromethylphenyl)-1(2H)-pyridineacetic acid ethyl ester

A mixture of 3-(4-trifluoromethylphenyl)-2(1H)-pyridinone (450 mg, 1.9 mmol) in dry DMF (10 mL) was treated with sodium hydride (90 mg, 3;7 mmol) at 0° C. The cooling bath was immediately removed. After stirring at room temperature for 45 min ethyl α-bromophenyl acetate (550 mg, 2.3 mmol) was added and the mixture stirred at room temperature over night. The mixture was partitioned between EtOAc and 1M HCl and the organic layer was washed with brine, dried (MgSO₄), and concentrated. The mixture was purified by column chromatography (EtOAc:heptane; 1:2) to provide a yellow solid, which was crystallized, from EtOAc and heptane to give 2-oxo-α-phenyl-3-(4-trifluoromethylphenyl)-1(2H)-pyridineacetic acid ethyl ester (588 mg, 78%).

¹H NMR (400 MHz, CDCl₃) δ 1.29 (t, J=7.3 Hz, 3 H), 4.32 (q, J=7.3 Hz, 2H), 6.24 (t, J=7.3 Hz, 1 H), 6.76 (s, 1 H), 7.12 (dd, J=7.3, 2.0 Hz, 1 H), 7.36-7.51(m, 5 H), 7.54 (dd, J=6.8, 2.0 Hz, 1 H), 7.67 (d, J=8.3 Hz, 2 H), 7.84(d, J=8.3 Hz, 2 H).

### Example 2

### 2-Oxo-α-phenyl-3-(4-trifluoromethylphenyl)-1(2H)-pyridineacetic acid

To a mixture of 2-oxo-α-phenyl-3-(4-trifluoromethylphenyl)-1(2H)-pyridineacetic acid ethyl ester (557 mg, 1.4 mmol) in dioxan (6 mL) and water (1 mL) was lithium hydroxide (65 mg of monohydrate, 2.0 mmol) added at 40° C. The mixture was stirred 40° C. at for additional 1 h before an additional portion of water (3 mL) added and the stirring continued at room temperature over night. The mixture was concentrated, acidified with 2M HCl, and the resultant cloudy mixture was extracted with twice with EtOAc. The combined organic layers was washed with brine, dried (MgSO₄), and concentrated. Crystallization from EtOAc and heptane gave 2-oxo-α-phenyl-3-(4-trifluoromethylphenyl)-1(2H)-pyridineacetic acid (0.465 g, 90%) as colorless solid.

¹H NMR (300 MHz, CDCl₃) δ 6.30 (t, J=7.0 Hz, 1 H), 6.63 (s, 1 H), 7.09 (dd, J=7.0, 2.0 Hz, 1 H), 7.30-7.50 (m, 5 H), 7.56 (dd, J=13.0, 2.0 Hz, 1 H), 7.65 (d, J=8.4 Hz, 2 ), 7.78 (d, J=8.1 Hz, 2 H).

### Example 3

### 1-[2-(4-Morpholinyl)-2-oxo-phenylethyl]-3-(4-trifluoromethylphenyl)-2(1H)-pyridinone

A mixture of 2-α-phenyl-3-(4-trifluoromethylphenyl)-1(2H)-pyridineacetic acid (125 mg, 0.33 mmol) in DM (4 mL) was treated with TBTU (118 mg, 3.7 mmol) followed by 4-morpholine (0.146 g, 1.7 mmol) and the mixture stirred at room temperature over for 62 h. The mixture was diluted with EtOAc, washed successively with saturated NaHCO₃ and brine, dried (MgSO₄), concentrated, and purified by column chromatography (EtOAc:heptane; 2:1) to provide 1-[2-(4-morpholinyl)-2-oxo-phenylethyl]-3-(4-trifluoromethylphenyl)-2(1H)-pyridinone (100 mg, 68%) as a colorless oil.

¹H NMR (400 MHz, CDCl₃) δ 3.30-3.39 (m, 2 H), 3.56-3.72 (m, 4 H), 3.74-3.86 (m, 2H), 6.24 (t, J=6.8 Hz, 1 H), 7.18 (dd, J=7.3,2.0 Hz, 1 H), 7.38-7.54 (m, 6 H), 7.66 (d, J=8.8 Hz, 2 H), 7.82 (d, J=8.3 Hz, 2 H).

### Example 4

### 1-[2-(4-Morpholinyl)-1-phenylethyl]-3-(4-trifluoromethylpheny))-2(1H)-pyridinone

1-[2-(4-Morpholinyl)-2-oxo-phenylethyl]-3-phenyl-2(1H)-pyridinone (0.100 g, 0.23 mmol) was dissolved in dry THF (4.0 mL) and borane-dimethyl sulfide complex (2 mL of 2M solution in ether, 4.0 mmol) was added at room temperature under nitrogen atmosphere. After stirring at room temperature over night the mixture was treated with methanol and 2M HCl. The mixture was concentrated and azeotroped twice with ethanol before it was purified by column chromatography (EtOAc:heptane:ammonia; 60:40:1 to 80:20:1) to provide the product as colorless oil. The pure product was taken up in Et₂O and HCl gas was bubbled through the solution. Filtration and lyphilozation from water gave the 1-[2-(4-morpholinyl)-1-phenylethyl]-3-phenyl-2(1H)-pyridinone (29 mg, 28%) as the corresponding HCl salt.

¹H NMR (400 MHz, CDCl₃) δ 2.52 (m, 2 H),2.69 (m, 2 H), 2.97 (s, 1 H), 3.05 (m, 1H), 3.58-3.68 (m, 4 H), 6.28 (t, J=6.8 Hz, 1 H), 6.60 (t, J=6.8 Hz, 1 H), 7.26 -7.40 (m, 6 H), 7.66 (m, 2 H), 7.86 (m, 2 H).

### Example 5

### 1-(2-Hydroxy-2-phenylethyl)-pyrrolidine

Styrene oxide (24 g, 0.2 mol) and pyrrolidine (21.3 g, 0.3 mol) were heated together under reflux temperature for 3 h. The mixture was then distilled to provide yellow oil, which solidified when standing. The product was purified by crystallization from heptane to provide 1-(2-hydroxy-2-phenylethyl)-pyrrolidine (21.6 g, 57%) as a colourless solid.

¹H NMR (400 MHz, CDCl₃) δ 1.84 (m, 4 H), 2.55 (m, 3H), 2.80 (m, 3 H), 4.74 (dd, J=10.3, 2.9 Hz; 1 H), 7.26-7.44 (m, 5 H).

### Example 6

### 1-(2-Chloro-2-phenylethyl)-pyrrolidine

To a vigorously stirred solution of thionyl chloride (3.6 g, 30 mmol) in Et₂O (30 mL) at 0° C. was α-phenyl-1-pyrrolidine ethanol (4.8 g, 25 mmol) in Et₂O (30 mL) added dropwise. The cooling bath was removed and the stirring was continued at room temperature for 2 h. The resulting tan precipitate was filtered and washed well with Et₂O. The product was purified by recrystallization-from acetonitrile. (5.4 g, 87%) and the pure product was partitioned between EtOAc and saturated NaHCO₃. The organic layer was washed with brine, dried (MgSO₄) and concentrated to give clean free 1-(2-bromo-2-phenylethyl)-pyrrolidine that was used directly in the next step.

¹H NMR 400 MHz, CDCl₃) δ 1.80 (m, 4 H), 2.60 (m, 4 H), 2.92 (dd, J=13.2, 5.9 Hz, 1 H), 3.23 (dd, J=13.2, 8.3 Hz, 1 H), 4.96 (dd, J=8.3, 5.9 Hz, 1 H), 7.30-7.46 (m, 5 H).

### Example 7

### 1-[1-Phenyl-2-(1-pyrrolidinyl)-ethyl]-3-[4-(trifluoromethyl)phenyl]-2(1H)-pyridinone

This was prepared from 3-(4-trifluoromethylphenyl)-2(1H)-pyridinone and 1-(2-chloro-2-phenylethyl)-pyrrolidine according to the procedure similar to that described in Example 1. The product was purified by column chromatography (EtOAc:heptane:arnrmonia; 50:50:0.5,) to provide the product as a colorless oil that was taken up in Et₂O and treated dropwise with a saturated solution of HCl in Et₂O to give 1-[2-(1-pyrrolidinyl)-1-phenylethyl]-3-(4-trichlorofluoromethylphenyl)-2(1H)-pyridinone as the corresponding HCl salt (0.26 g, 58%).

¹H NMR (300 MHz, CDCl₃) δ 1.70 (m, 4 H); 2.60 (m, 2 H), 2.66 (m, 2H), 3.10 (dd, 1H), 3.24 (dd, 1 H), 6.24 (t, 1 H), 6.50 (dd, 1 H), 7.22-7.44 (m, 6 H), 7.50 (dd, 1 H), 7.68 (d, 2 H), 7.86 (d, 2 H).

### Example 8

### 4-Phenyl-2-[1-phenyl-2-(1-pyrrolidinyl)ethyl]-3(2H)-pyridazinone

This was prepared from 4-phenyl-3(2H1)-pyridazinone and 1-(2-chloro-2-phenylethyl)-pyrrolidine according to a procedure similar to that described in Example 1. The product was purified by column chromatography (EtOAc:heptane; ammonia; 50:50:0.5) to provide the product as a colorless oil that was taken up in Et₂O and treated dropwise with a saturated solution of HCl in Et₂O to give 4-phenyl-2-[1-phenyl-2-(1-pyrrolidinyl)ethyl]-3(2H)-pyridazinone as the corresponding HCl salt (0.028 g, 19%).

¹H NMR (300 MHz, CDCl₃) δ 1.73 (m, 4 H), 2.56 (m, 2 H), 2.70 (m, 2 H), 2.95 (dd, J=12.4, 5.0 Hz, 1 H), 3.37 (dd, J=12.4, 10.0 Hz, 1 H), 6.55 (dd, J=9.7, 5.0 Hz, 1 H), 7.70-7.58 (m, 9 H), 7.78 (m, 2 H), 7.90 (d, J=4.0 Hz, 1 H).

### Example 9

### 1-[2-Hydroxy-2-(2-methoxy-phenyl)-ethyl]pyrrolidine

A mixture of pyrrolidine (6.3 mL, 75 mmol) in THF (20 mL) was added dropwise to a solution of 2-bromo-2′-methoxyacetophenone (3.4 g, 15 mmol) in THF (20 mL) at room temperature. After 2 h stirring at room temperature the reaction mixture was concentrated and re-solvated in MeOH (40 mL) before sodiumborohydride (1.13 g, 30 mmol) was added. The reaction mixture was stirred at room temperature for 2 h, concentrated, and finally partitioned between CH₂Cl₂ and water. The organic layer was washed with brine, dried (MgSO₄) and concentrated to give clean free 1-[2-hydroxy-2-(2-methoxy-phenyl)-ethyl]pyrrolidine (2.26 g, 68%) that was used directly in the next step.

¹H NMR

### Example 10

### 1-[1-(2-Methoxyphenyl)-2-(1-pyrrolidinyl)-ethyl]-3-[4-(trifluoroinethyl)phenyl)-2(1H)-pyridinone) and

### 2-[1-(2-Methoxyphenyl)-2-(2-pyrrolidinyl)-ethyl]-3-[4-(trifluoromethyl)phenyl)-pyridine

To a mixture of 3-(4-trifluoromethylphenyl)-2(1H)-pyridinone, (28.7 mg, 0.12 mmol), tributylphosphine (26.3 mg, 0.13 mmol) and 1-[2-hydroxy-2-(2-methoxy-phenyl)-ethyl] pyrrolidine (22.1 mg, 0.10 mmol) in dry THF (1.5 mL) was a solution of 1,1′-azobis-(NN-dimethylformamide) (22. 4 mg, 0.13 mmol) in dry THF (0.5 mL) slowly added at room temperature. The mixture was stirred for 14 h at room temperature and then filtered to remove the tributylphosphineoxide formed during the reaction. The mixture of the N- and O-alkylated products was purified by using an isolute SCX-SPE column (THF, CH₂Cl₂, MeOH, to MeOH saturated with NH₃). The two products were separated by preparative HPLC to provide 1-[1-(2-methoxyphenyl)-2-(1-pyrrolidinyl)-ethyl]-3-[4-(trifluoromethyl)phenyl)-2(1H)-pyridinone) (0.020 g, 45%) and 2-[1-(2-methoxyphenyl)-2-(1-pyrrolidinyl)-ethyl]-3-[4-(trifluoromethyl)phenyl)-pyridine (2.7 mg, 5%). 1-[1-(2-methoxyphenyl)-2-(1-pyrrolidinyl)-ethyl]-3-[4-(trifluoromethyl)phenyl)-2(1H)-pyridinone): ¹H NMR (400 MHz, CDCl₃) δ 1.72 (m, 4 H), 2.61 (m, 2 H), 2.70 (m, 2 H), 3.13 (dd, J=13.7, 6.8 Hz, 1 H), 3.28 (dd, J=13.7, 8.2 Hz, 1 H), 3.82 (s, 3 H), 6.25 (t, J=6.8 Hz, 1 H), 6.51 (t, J=4.2 Hz, 1 H), 6.89 (d, J=8.3 Hz, 1 H), 6.97 (t, J=4.2 Hz, 1 H), 7.40-7.50 (m, 4 H), 7.62 (d, J=8.3 Hz, 2 H), 7.82 (d, J=8.3 Hz, 2 H).

2-[1-(2-methoxyphenyl)-2-(1-pyrrolidinyl)-ethyl]-3-[4-(trifluoromethyl)phenyl)-pyridine:

¹ H NMR (400 MHz, CDCl₃) δ 1.56 (m, 4 H), 2.50 (m, 2 H), 2.60 (m, 2 H), 3.67 (s, 3 H), 4.23 (dd, J=6.8, 5.2 Hz, 1 H), 4.61 (J=10.2, 7.3 Hz, 1 H), 4.76 (dd, J=9.5, 4.9 Hz, 1 H), 6.85-6.98 (m, 4 H), 7.30-7.58 (m, 6 H), 8.17 (m, 1 H).

### Example 11

### 1-[1-Phenyl-2-(1-pyrrolidinyl)-ethyl]-3-bromo-2(1)-pyridinone

This was prepared from 3-bromo-2(1H)-pyridinone and 1-(2-chloro-2-phenylethyl)-pyrrolidine according to the procedure similar to that described in Example 1. The product was purified by column chromatography (CH₂Cl₂:MeOH:ammonia; 100:1:0.5,) to provide the product as a yellow solid that was first crystallized from EtOAc and heptane, and then taken up in Et₂O and treated dropwise with a saturated solution of HCl in Et₂O to give 1-[1-phenyl-2-(1-pyrrolidinyl)-ethyl]-3-bromo-2(1H)-pyridinone as the-corresponding HCl salt (2.95 g, 59%).

¹H NMR (300 MHz, CDCl₃) δ 1.67 (m, 4 H), 2.46 (, 2 H), 2.60 (m, 2 H), 3.00 (dd, J=10.5, 7.0 Hz, 1 H), 3.20 (dd, J=14.0, 7.0 Hz, 1 H), 6.02 (t, J=7.3 Hz, 1 H), 6.36 (dd, J=10.5, 5.0 Hz, 1 H), 7.20-7.35 (m, 6 H), 7.62 (dd, J=7.5, 2.5 Hz, 1 H).

### Example 12

### 1-[-Phenyl-2-(1-pyrrolidinyl)-ethyl]-3-(3,4-dichlorophenyl)-2(1H)-pyridinone

To a mixture of 1-[1-phenyl-2-(1-pyrrolidinyl)-ethyl]-3-bromo-2(1H)-pyridinone (0.060 g, 0.17 mmol), 3,4-dichloroboronic acid (1 mL of a 50% solution in THF:H2O, 0.26 mmol), and potassium carbonate (0.060 g, 0.43 mmol) in a 1:1 mixture of acetone:water (4 mL) was PdOAc₂ (0.8 mL of a 8 mM solution in acetone, 0.006 mmol) added. The reaction mixture was heated at 70° C. for 1 h before it was cooled and concentrated to remove the acetone. The remaining mixture was diluted with saturated NaHCO₃ and extracted with EtOAc. The EtOAc extract was washed with brine, dried, and concentrated. Purification by column chromatography (EtOAc:heptane:ammonia; 60:40:0.5 to 75:25:0.5) gave the product as a colorless oil. The product was taken up in Et₂O and treated dropwise with a saturated solution of HCl in Et₂O to give 1-[1-phenyl-2-(1-pyrrolidinyl)-ethyl]-3-(3,4-dichlorophenyl)-2(1H)-pyridinone as the corresponding HCl salt (0.042 g, 54%).

¹H NMR (400 MHz, CDCl₃) δ 1.78 (m, 4 H), 2.78 (m, 2 H), 2.89 (m, 2 H), 3.26 (dd, J=12.7, 5.4 Hz, 1 H), 3.46 (dd, J=12.7, 8.8 Hz, 1 H), 6.24 (t, J=6.8 Hz, 1 H), 6.43 (dd, J=9.3, 5.9 Hz, 1 H), 7:21-7.54 (m, 9 H), 7.82 (d, J=2.0 Hz, 1 H).

### Example 13

### 1-[1-Phenyl-2-(1-pyrrolidinyl)-ethyl]-S-bromo-2(1H)-pyridinone

This was prepared from 5-bromo-2(1H)-pyridinone and 1-(2-chloro-2-phenylethyl)-pyrrolidine in 42% yield according to the procedure similar to that described in Example 1.

¹H NMR (400 MHz, CDCl₃) δ 1.70 (m, 4 H), 2.52 (m, 2 H), 2.64 (m, 2 H), 3.02 (dd, J=13.2, 6.3 Hz, 1 h), 3.21 (dd, J=13.2, 9.8 Hz, 1 H), 6.33 (dd, J=9.3, 5.9 Hz, 1 H), 6.50 (d, J=9.8 Hz, 1 H), 7.24-7.38 (m, 7 H).

### Example 14

### 1-[1-Phenyl-2-(1-pyrrolidinyl)ethyl]-S-(3,4-dichlorophenyl)-2(1H)-pyridinone

To a mixture of 1-[1-phenyl-2-(1-pyrrolidinyl)-ethyl]-5-bromo-2(1H)-pyridinone (0.080 g, 0.23 mmol), 4-chloroboronic acid (0.046 g, 0.29 mmol) in a 1:1 mixture of THF: saturated NaHCO₃(4 mL) was Pd(PPh₃)₄ (0.008 g, 0.007 mmol) added. The reaction mixture was heated under reflux for 4.5 h before it was partitioned between saturated brine and EtOAc. The organic layer was dried (Na₂SO₄) and concentrated. Purification by column chromatography (EtOAc:heptane:ammonia; 75:25:1) gave the product as a colourless oil. The product was taken up in Et₂O and treated dropwise with a saturated solution of HCl in Et₂O to give 1-(1-phenyl-2-(1-pyrrolidinyl)-ethyl]-5-(4-chlorophenyl)-2(1H)-pyridinone as the corresponding HCl salt (0.043 g, 45%).

¹H NMR (MHz,).

### Example 15

### 5-Bromo-[1-phenyl-2-(1-pyrrolidinyl)ethyl]-4(3H)-pyrimidinone

This was prepared from 5-bromo-4(3H)-pyrimidinone and 1-(2-chloro-2-phenylethyl)-pyrrolidine according to the procedure similar to that described in Example 1. The product was purified by column chromatography (CH₂Cl₂, MeOH: ammonia; 100:1:0.5,) to provide 5-bromo-[1-phenyl-2-(1-pyrrolidinyl)ethyl]4(3H)-pyrimidinone ( 0.26 g, 67%) as a colourless solid.

¹H NMR (400 MHz, CDCl₃) δ 1.72 (m, 4 H), 2.51 (m, 2 H), 2.61 (m, 2 H), 2.95 (dd, J=13.2, 5.4 Hz, 1 H), 3.39 (dd, J=13.2, 10.7 Hz, 1 H), 6.11 (dd, J=10.3, 5.4 Hz, 1 H), 7.12 (m, 5 H), 8.16 (s, 1 H), 6.17 (s, 1 H).

### Example 16

### 5-(3,4-Dichlorophenyl)-[1-phenyl-2-(1-pyrrolidinyl)ethyl)-4(3H)-pyrimidinone

This was prepared from 5-bromo-[1-phenyl-2-(1-pyrrolidinyl)ethyl]-4(3H)-pyrimidinone and 3,4-dichlorophenylboronic acid according to a procedure similar to that described in Example 13. The product was purified by column chromatography (CH₂Cl₂, MeOH: ammonia; 75:25:1) to provide the product as a colourless oil that was taken up in Et₂O and treated dropwise with a saturated solution of HCl in Et₂O to give 5-(3,4-dichlorophenyl)-[1-phenyl]-2-(1-pyrrolidinyl)ethyl]-4(3H)-pyrimidinone as the corresponding HCl salt (0.068 g, 66%).

¹H NMR (400 MHz, CDCl₃) δ 1.74 (m, 4 H), 2.56 (m,2 H), 2.65 (m, 2 H),3.00 (dd, J=13.2, 5.4 Hz, 1 H), 3.41 (dd, J=13.2, 10.7 Hz, 1 H), 6.20 (dd, J=10.7, 5.4 Hz, 1 H), 7.30-7.73 (m, 7 H), 7.75 (d, J=2.0 Hz, 1 H), 8.05 (s, 1H), 8.26 (s, 1 H).

### Example 17

### 3-[(4-Methylphenyl)amino]-1-[1-phenyl-2-(1-pyrrolidinyl)ethyl]-2(1H)-pyridinone

An oven-dried flask was cooled to room temperature under an argon atmosphere and charged with 1-[1-phenyl-2-(1-pyrrolidinyl)-ethyl]-3-bromo-2(1H)-pyridinone (0.20 g, 0.58 mmol), toluidine (0.075 g, 0.70 mmol), Pd₂(dba)₃ (0.003 g, 0.003 mmol), R-BIAP (0.005 g, 0.008 mmol), and potassium tert-butoxide (0.077 g, 0.80 mmol) followed by dry dioxane (2.0 mL). The reaction mixture was stirred at 90° C. for 18 h under an argon atmosphere. The reaction mixture was cooled to room temperature, diluted with EtOAc, filtered, and concentrated. Purification by column chromatography (EtOAc:Heptane: NH₄OH; 10:10: 0.3) gave the product as a slightly green solid. The product was taken up in Et₂O and treated dropwise with a 4M solution of HCl in dioxane to give 3-[(4-methylphenyl)amino]1-[1-phenyl-2-(1-pyrrolidinyl)ethyl]-2(1H)-pyridinone as the corresponding HCl salt (0.16 g, 66%).

¹H NMR (400 MHz, CDCl₃) δ 1.75 (m, 4 H), 2.33 (s, 3H), 2.59 (m, 2 H), 2.67 (m, 2 H), 3.12-3.27 (m, 2 H), 6.13 (t, J=7.3 Hz, 1 H), 6.47 (dd, J=7.3, 5.8 Hz, 1 H), 6.78 (dd, J=6.8, 1.5 Hz, 1H), 6.94 (dd, J=7.3, 1.5 Hz, 1 H), 7.10-7.16 (m, 4 H), 7.26-7.42 (m, 5 H).

General Procedure for the Solid-Phase Synthesis of the Compound 1.

To a round-bottom flask was added under an argon atmosphere polystyrene-diethylsilane (PS-DES) resin (4.0 g, 1.35 mmol/g) followed by a solution of 2-hydroxypyrrolidine (0.15 M)) and RhCl(PPh₃)₃ (3.0 mM) in dry NMP (10 mL/g). After shaking at 60° C. for 2 h, the resin was isolated by filtration and rinsed with NMP (×3), CH₂Cl₂ (×3), and THF (×3). The resin was then reacted with styreneoxide (30 mL) at 60° C. for 6 h before it was isolated by filtration and rinsed with DMF (×3), CH₂Cl₂ (×3), and MeOH (×3). In the next step the resin was treated with a mixture of 3-bromo-2(1H)-pyridinone, (0.06 M), tributylphosphine (0.065 M) in dry THF (48 mL) followed by a solution of 1,1′-azobis-(N,N-dimethylformamide) (0.065 M) in dry THF (16 mL) and the resulting mixture was shakened for 14 h at room temperature. The resin was isolated by filtration and rinsed with THF (×3), DMF (×3), CH₂Cl₂ (×3), and MeOH (×3). The resin (0.035 g, 1.35 mmol/g) was solvated in DMF (14 mg of resin) and K₂CO₃ (0.5 M), Pd(PPh₃)₄ (0.005 M), and the arylboronic acid (0.2 M) were added. After 24 h shaking at 85° C., the resin was collected by filtration and rinsed with CH₂Cl₂ (×3), DMF (×3), H₂O (×3), THF (×3), and Et₂O (×3). The desired product was cleaved from the resin by treatment with AcOH:THF:H₂O (6:6:1) (0.5 mL) for 14 h at 80° C. The solution was filtered to remove the resin and the filtrate was concentrated to give the crude product, which was purified by preparative HPLC to provide pure compound 1.

Receptor Binding

Compounds of the invention, prepared using the general methods described above, may be tested for their ability to bind to the human kappa opioid receptor using standard procedures well known to those skilled in the art.

Examples of standard procedures are given below.

### Binding Assay for Opioid Receptor Transfected Cell Membrane in 96-Well Format

Principle: The procedures describe how to perform and interpret receptor binding experiments designed to determine the affinities of new compounds for μ, δ and κ receptors. The total and nonspecific binding are determined in the absence and presence of selective opioid receptor radiolabeled ligand.

Procedure for Kappa Opioid Receptor:

The total and non-specific binding are determined in absence and presence of 1 μM of Asimadoline and added with the multichannel pipett in respectively well of the 96 well plate (see table 1).

The radioligand 125I-(D-pro)-Dynorphin A (1-1 1) is added containing 30 000-50 000 CPM per well.

Membranes are thawed at room temperature and diluted in assay buffer to the appropriate amount of protein, homogenized 5-10 times in glas/glas, and added to the 96 well plate.

Vortex the plate 1 minute, incubate at room temperature for 60-75 minutes, vortex 1 minute and the reaction was terminated by rapid filtration (Micro96 Harvester, Skatron Instruments, Norway) to separate bound from free ligand. The filters used (Filtermat A or B, Wallac, Finland) were presoaked in 0.1% polyetylenimine (PEI) for 10 minutes and then dried 1 hour, 50° C. After filtration the filters were dried and a scintillator sheet (Meltilex A or B/HB, Wallac, Finland) was melted on the filter (microsealer 1495-021, Wallac, Finland).

The amount of bound ligand was measure by counting for β-radiation in a β-liquid scintillation counter (1450 Microbeta TRILUX, Wallac, Finland). The results were presented as decomposition per minute (DPM).

Percentage inhibition is calculated for each well from the two standards, 0% and 100% inhibition.

Solutions: Assay Buffer:

The binding buffer, 50 mM Tris, 3 mM MgCl₂, stored at +4° C.

Freshly made with 0.1% BSA, at day for experiment.

Membrane:

Aliquots of membranes are stored in −70°, diluted in buffer to a final concentration of 10 μg/sample.

Ligand:

Asimadoline, 200 μM in DMSO.

Radioligand:

¹²⁵I-(D-Pro¹⁰)-Dynorphin A (1-11)

Substance-Plates:

80 substances dissolved in pure DMSO were kept in the 96 well plate format. The remaining 24 wells i.e. column no 1 and 12 contained pure DMSO and were used for the assay standard and control wells. The stock compound concentration was 10 or 3 mM. Within a run, the compounds were diluted 200 times which gave an assay concentration of 10 μM.

This gave a DMSO concentration of 0.5%. Effects on the assay of DMSO occur, using concentrations of DMSO over 0.5%.

Standards and Control Wells:

Wells in column 1 and 12 are used as an assay control and for percentage inhibition calculations:

Standard 1, 0% inhibition, total activity. The total binding are determined in absence of 1 μM of Asimadoline.

Standard 2, 100% inhibition, non-specific binding are determined in presence of 1 μM of Asimadoline.

The control, which gives about 50% inhibition, is a known inhibitor (Asimadoline, IC₅₀-value about 4-10 nM) dissolved in DMSO.

Counter: Wallac TRILUX MicroBeta.

Assayplate: Nunc™ Brand Products, Nalge Nunc International, 96 well sample plate.

Chemicals: Ligand: Asimadoline

Radioligand: ¹²⁵I-(D-Pro¹⁰)-Dynorphin A (1-11).

Euro-Diagnostica AB, Malmö, Sweden.

Enzyme: Membrane preparations from the HEK293S KOR cells has been prepared by ABL. Storage −70° C.

Buffer: Trizma Base (Tris[hydroxymethyl]aminomethane)

Mw121.1 (Sigma #T-8404)

Magnesiumchlorid Hexahydrat p.a., Mw 203,30 (Merck #1.05833)

BSA (fraction V, Sigma # A-6793)

DMSO p.a. (Merck #1.6069-1)

### SPA Binding Assay for Kappa-Opioid Receptor Transfected Cell Membrane in 96-Well Format

Principle: Membranes containing the kappa opioid receptor are coupled to SPA beads, the ability of tested compounds to dislocate a radio ligand the protein agonist Dynorphin. (¹²⁵I-(D-Pro¹⁰)-Dynorphin A (1-11)) are determined. Only radioaktivity in close proximity to the beads, that is bound to membranes attached to beads, will be measured.

Procedure: The total and non-specific binding are determined in absence and presence of 0.5 μM of Dynorphin, radioligand is added containing 15 000-20 000 CPM per well.

The membrane and SPA beads precouples over night (18-22 hours) in assay buffer at room temperature on a shaking platform, with rotary movement. The mixture of membrane and beads are pipetted together with the assay-buffer, agents and the radioligand into a 96-well microtiterplate.

The plate is incubated for 1 hours at room temperature, and counted in a WALLAC TRILUX MicroBeta.

Percentage inhibition is calculated for each well from the two standards, 0% and 100% inhibition.

Solutions: Assay Buffer:

The binding buffer, 50 mM Tris, 3 mM MgCl₂, stored at +4° C.

Freshly made with 0.1% BSA,at day for experiment.

Membrane:

Aliquots of membranes are stored in −70°, diluted in buffer to a final concentration of 10 μg/sample.

SPA Beads:

SPA beads are reconstituted in distilled water to a concentration of 100 mg/ml. This solution can be stored at 4° C. up to one week. Prior to use, the beads are diluted in assay buffer to a final amount of 0.1 mg beads per well.

Precoupling of Beads to Membrane:

Equal volumes of bead and cell-membrane suspensions are gently stirred at room temperature 18-22 hours before the experiment start.

Ligand:

Dynorphin A (1-17)(Porcine)*8 AcOH*8 H₂O, 200 μM in bindingbuffer without BSA, stored in aliquots at −20° C.

Diluted to final concentration (fresh) to each experiment.

Radioligand: ¹²⁵I-(D-Pro¹⁰)-Dynorphin A (1-11)

Substance-plates: 80 substances dissolved in pure DMSO were kept in the 96 well plate format. The remaining 24 wells i.e. column no 1 and 12 contained pure DMSO and were used for the assay standard and control wells. The stock compound concentration was 0.6 mM. Within a run, the compounds were diluted 200 times which gave an assay concentration of

3 μM. This gave a DMSO concentration of 1.4%. Effects on the assay of DMSO occur, using concentrations of DMSO over 0.5%.

Standards and Control wells: Wells in column 1 and 12 are used as an assay control and for percentage inhibition calculations:

Standard 1, 0% inhibition, total activity. The total binding are determined in absence of 0.5 μM of Dynorphin.

Standard 2, 100% inhibition, non-specific binding are determined in presence of 0.5 μM of Dynorphin.

The control, which gives about 50% inhibition, is a known inhibitor (Asimadoline, IC₅₀ -value about 4 nM) dissolved in DMSO.

A specially designed trough was used where the pipetting of the standard 1, standard 2 and the control is kept separately.

Robotic system: SAIGAN™ Core System from Beckman Coulter

Pipetting system: Multimek, 200 μl pipetting head and 50 μl Robbins-tips

Counter: Wallac TRILUX MicroBeta

Assayplate: Isoplate™ polystyrene (ps) well, 96 well Rigid sample plate for use with 1450 MicroBeta.

Chemicals: Ligand: Dynorphin A (1-17)(Porcine)*8 AcOH*8 H₂O.

Bachem Feinchernikalien AG, Switzerland.

Radioligand: ¹²⁵I-(D-Pro¹⁰)-Dynorphin A (1-11).

Euro-Diagnostica AB, Malmö, Sweden.

Enzyme: Membrane preparations from the HEK293S KOR cells has been prepared by ABL. Storage −70° C.

Buffer: Trizma Base (Tris[hydroxymethyl]aminomethane)

Mw121.1 (Sigma #T-8404)

Magnesiumchlorid Hexahydrat p.a., Mw 203,30 (Merck #1.05833)

BSA (fraction V, Sigma # A-6793)

DMSO p.a. (Merck #1.6069-1)

SPA beads: Wheatgerm agglutinin SPA beads RPNQ 0001 (Amersham LIFE SCIENCE)

1. A compound of formula (1) having the following structure: 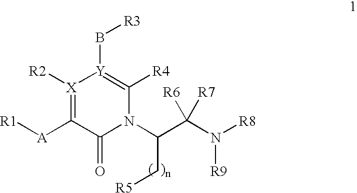
wherein
X and Y are independently C or N;
A is a direct bond, CH₂ or NH;
B is a direct bond or NH;
n=0-2;
R1 is H, optionally substituted C₁₋₄ alkyl, C₃₋₇ cycloalkyl, halogen, cyano, nitro, aryl, or alkylaryl;
R2 is H, C₁₋₄ alkyl, or alkoxy C₁₋₄ alkyl; or
R1 and R2 are taken together to form an unsaturated heterocyclic ring containing one or two heteroatoms fused to the pyridone;
R3 is H, C₁₋₄ alkyl, substituted C₁₋₄ alkyl, C₃₋₇ cycloalkyl, aryl or alkylaryl;
R4 is H;
R5 is C₁₋₄ alkyl or aryl;
R6 and R7 are independently H or C₁₋₄ alkyl;
R8 and R9 are independently H, C₁₋₄ alkyl, or tert-butoxycarbonyl or
R8 and R9 are taken together with the nitrogen to which they are attached and form optionally, unsubstituted, substituted, fused or unsaturated 5-,6-,7-membered heterocycles containing one or two heteroatoms wherein said substituents are selected from the group consisting of hydroxyl, hydroxymethyl, carboxymethyl, carboxy, methoxy, and tert-butoxy; or
a compound of formula (2) having the following structure: 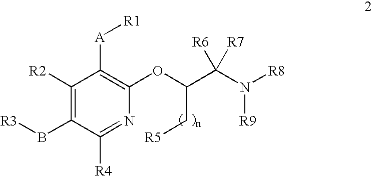
wherein
A, B, n, R3, R4, R5, R6, R7, R8 and R9 are as defined above and
R1 is H, optionally substituted C₁₋₄alkyl, C₃₋₇ cycloalkyl, halogen, cyano, nitro, aryl, or alkylaryl;
R2 is H, C₁₋₄ alkyl, or alkoxy C₁₋₄ alkyl; or
R1 and R2 are taken together to form an unsaturated 6-membered aromatic or heterocyclic ring containing one or two heteroatoms fused to the pyridone; or
a compound of formula (3) having the following structure: 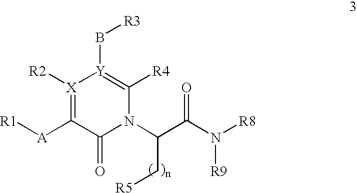
wherein
A, B, X, Y, n, R1, R2, R3, R4, R5, R8, and R9 are as defined above and
R1 is H, optionally substituted C₁₋₄ alkyl, C₃₋₇ cycloalkyl, halogen, cyano, nitro, aryl, or alkylaryl;
R2 is H, C₁₋₄ alkyl, or alkoxy C₁₋₄ alkyl; or
R1 and R2 are taken together to form an unsaturated 6-membered aromatic or heterocyclic ring containing one or two heteroatoms fused to the pyridone;
as (R)-enantiomers, (S)-enantiomers or a racemate in the form of a free base or a pharmaceutically acceptable salt or solvate thereof.

2. A compound according to claim 1 characterized in that X and Y are C.

3. A compound according to any one of claims 1-2 characterized in that n=0.

4. A compound according to any one of claims 1-3 characterized in that R6 and R7 are H.

5. A pharmaceutical formulation comprising as active ingredient a therapeutically effective amount of the compound of any one of claims 1-4 as an enantiomer or racemate in the form of a free base or a pharmaceutically acceptable salt or solvate thereof optionally in association with diluents, excipients or inert carriers.

6. A compound as defined in any of claims 1-4 for use in therapy.

7. The use of a compound defined in any of claims 1-4 in the manufacture of a medicament wherein said compound is used as analgesic, anti-inflammatory, diuretic, antitussive, anesthetic or neuroprotective agents, or as agents for treatment of stroke or functional bowel disorders.
